(19) United States
(12) Patent Application Publication
(10) Pub. No.: US 2006/0264815 A1
(43) Pub. Date: Nov. 23, 2006

(54) Administering device comprising a protected primer

(76) Inventors: Edgar Hommann, Grossaffoltern (CH); Benjamin Scherer, Uster (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/406,812

(22) Filed: Apr. 19, 2006

(30) Foreign Application Priority Data
Apr. 20, 2005 (DE) 10 2005 018 305.0

(51) Int. Cl. A61M 5/30

(52) U.S. Cl. 604/68

(57) ABSTRACT

A device for administering, delivering or dispensing a substance, the device including a surface element forming at least portion of a surface of the device and forming or defining an opening, and a priming mechanism for priming the device, wherein the priming mechanism includes an activation element for activating a priming procedure, wherein the activation element is provided in the opening of the surface element in such a way that it is either flush with or inset relative to the surface of the surface element.

### CROSS-REFERENCE TO RELATED APPLICATION

This application claims the benefit of German Application No. 10 2005 018 305.0, filed on Apr. 20, 2005, the entire contents of which are incorporated herein by reference.

### BACKGROUND

The present invention relates to devices for administering, delivering or dispensing substances, and methods of making and using such devices. More particularly, it relates to an administering device, in particular an injection, infusion or inhalation device, for administering a substance, in particular a medical substance.

In the aforesaid and like devices, it is desirable to perform a so-called priming procedure. Before the substance—typically a fluid, in particular a liquid—is administered or delivered, it is desirable for the entire volume of the substance reservoir to be filled up with the substance to the outlet opening of the device. This ensures that the correct amount or dosage is delivered or administered during, for example, an injection procedure. It also ensures that undesirable quantities of air are not injected. In order to achieve this, the so-called priming procedure is performed, in which substance is delivered from the reservoir and ultimately also from the outlet opening (in an injection device, from the tip of the needle). This ensures that the substance fills up the entire volume of the reservoir, to the outlet opening of the injection device or administering device. In injection devices, the priming procedure is usually performed by means of a piston which presses the substance—in particular, a fluid—out of the reservoir by reducing the volume of the reservoir. Other priming methods and mechanisms, e.g., aspiration, rotational and/or translational mechanisms, pumps, linkages, rods, driving and driven members may be involved as well.

The priming procedure is usually triggered by an activation element which brings about or effects the displacement procedure by means of shifting the piston or actuates the priming mechanism appropriately. To this end, the activation element usually has to be pressed.

### SUMMARY

The present invention to provides for a priming mechanism for a device for dispensing, delivering or administering a substance, and an activation element for a device for administering, delivering or dispensing a substance, including for the priming mechanism, wherein the activation element minimizes the chance that it could be inadvertently activated and, therefore, minimizes inadvertent priming of the device.

In one embodiment, the present invention comprises a device for administering, delivering or dispensing a substance, the device comprising a surface element forming at least portion of a surface of the device and defining an opening, and a priming mechanism for priming the device, wherein the priming mechanism comprises an activation element for activating a priming procedure, wherein the activation element is provided in the opening of the surface element in such a way that it is either flush with or inset relative to the surface of the surface element.

In one embodiment, an administering device in accordance with the present invention advantageously prevents the activation element from being inadvertently activated, since the activation element does not protrude beyond an outer surface of the administering device and, more particularly, does not protrude from a surface element which forms an outer boundary of the administering device. The surface element can be a casing component or, for example, a setting ring for setting a dosage or amount to be delivered or administered. If the administering device is, for example, inadvertently dropped or wrongly grasped, the activation element cannot be inadvertently activated since it does not protrude beyond an outer surface of the casing.

The surface element is or forms a part of the outer surface of the administering device. The surface element can be a part of the casing of the administering device or, as described below by way of an example, can be a portion of a setting ring for setting a dosage to be administered. The surface element may define or comprise an opening in which the activation element for activating priming is located. The activation element is a component of a priming mechanism or means which is formed such that when it is activated, i.e., when the activation element is pressed or manipulated, it causes a rod to shift within the administering device. The rod then presses on a piston which displaces a substance and/or fluid in a reservoir of the administering device. Generally, the reservoir contains the substance to be administered.

In order to prevent the activation element from being inadvertently activated, in some preferred embodiments, it is formed such that it does not protrude or extend outwardly out of the opening of the surface element, wherein “outwardly” means towards and/or past the outside of the administering device. In some embodiments, the surface of the activation element pointing outwardly may be flush with the surface of the surface element and, in other embodiments it may be inset or sunk relative to the surface of the surface element, in other words, being inside or lower than the exterior or outside surface of the surface element or the administering device.

In accordance with the invention, the aforesaid arrangement is already provided when the activation element has not yet been activated and a priming procedure before the next administering has not yet been performed.

In some preferred embodiments, the surface element is formed in the shape of a generally hollow cylinder or sleeve, generally at the rear (or proximal) end of the administering device. The activation element is configured such that it can be shifted, in the interior of the sleeve, in the longitudinal direction of the sleeve, in the forward direction (i.e., the distal direction or toward the front or substance emitting end), in order to effect the priming procedure. The activation element can of course also be arranged at a different point on the administering device, e.g., on the longitudinal side.

In some preferred embodiments, the activation element is configured such that it comprises an engaging section via which it engages with a priming rod. To this end, the activation element can comprise a sleeve which points inwards and surrounds the priming rod. Because the activation element is engaged with the priming rod, moving the activation element inwardly moves the priming rod, which in turn moves a piston which displaces air and/or the substance in the reservoir.

In order to configure the administering device to be compact, in some embodiments the surface element is a setting ring formed for setting a dosage to be administered, such as is described in detail in parallel application DE 10 2004 025 546.6-44. As described in DE 10 2004 025 546.6-44, a dosage can be set by turning the setting ring, wherein the setting ring is configured such that an accommodating space in which the activation element can be accommodated is at its end.

### BRIEF DESCRIPTION OF THE DRAWING

FIG. 1 shows an end of one embodiment of an administering device in accordance with the present invention.

### DETAILED DESCRIPTION

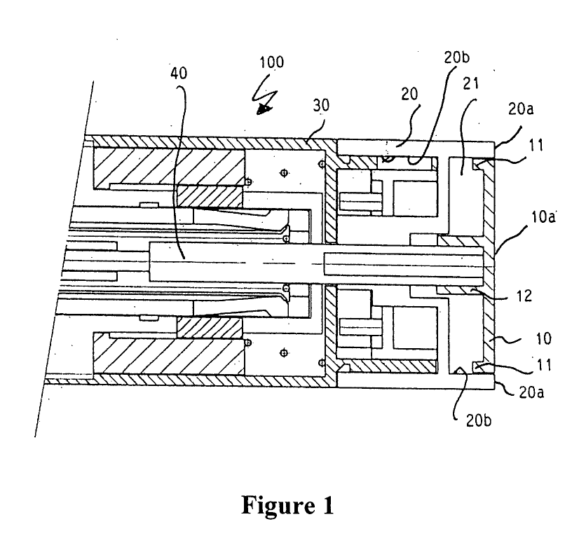

As can be seen in FIG. 1, in some embodiments, the activation element 10 is located at one end of a device for administering, delivering, dispensing or injecting, in the depicted instance, at the rear or proximal end. On the longitudinal side of the administering device 100, the activation element 10 is surrounded by a setting ring 20. The outer surface of the setting ring 20 is annular. At the end of the setting ring 20 (on the right-hand side in FIG. 1), the setting ring 20 forms a cylindrical cavity 21 which is open towards the rear end. The activation element 10 is fitted into this opening, such that it can be shifted longitudinally with respect to the longitudinal length of the administering device 100. The activation element 10 is formed in the shape of a disc, such that the generally flat, disc-shaped surface 10a of the activation element is flush with the proximal ends 20a of the setting ring 20. The surface element is, by way of an example, formed by the setting ring 20 and, in some preferred embodiments, is formed such that it is rigid and cannot be displaced in the longitudinal direction with respect to the casing 30. This protects the activation element 10 from being displaced into the interior of the administering device when a force is exerted on the ends 20a. This can prevent the device from being unintentionally primed, if, for example, the administering device unintentionally falls to the floor. In particular, the proximal ends 20a of the setting ring 20 annularly enclose the opening in which the activation element 10 is provided.

In some preferred embodiments, the opening for accommodating the activation element is provided on the end of the administering device. The activation element is fitted into the opening, in particular with a clearance. To this end, the activation element 10 comprises an annular continuation 11 extending or pointing toward the front or dispensing end of the device (i.e., in the distal direction). Said annular continuation 11 is formed such that it can slide along the inner surface of the setting ring 20, such that the activation element can be moved in the forward direction without being obstructed by the setting ring 20. The annular continuation 11 also serves as a guide for the activation element 10 along the sleeve-shaped or cylindrical inner wall 20b of the setting ring 20.

The activation element 10 also comprises a generally central, cylindrical, sleeve-shaped continuation 12 which is located on the inside surface of the button-like activation element, and radially inside the outer peripheral edge of the element 10, i.e., radially inside the outside annular continuation 11. The sleeve-shaped continuation 12 is a coupling hub for receiving and/or surrounding one end of a priming rod 40. If the activation element 10 in FIG. 1 moves to the left, i.e. distally, then the priming rod is also shifted in the distal direction. The priming rod 40 is connected to a piston (not shown) which is located in a reservoir (not shown). If the priming rod 40 is shifted in the distal direction, then the piston is also shifted and thus displaces fluid and/or substance out of the reservoir. In injection devices, the reservoir is connected to a hollow needle. The substance is thus injected outwards through the hollow needle by means of the piston when the activation element 10 is pressed in the distal direction. This can remove air which may be present from the reservoir, and the substance thus fills up both the reservoir and the needle, from the piston to the outlet end of the needle. A similar effect can be achieved in the fluid or dispensing pathway of other administering devices, such as infusion and inhalation devices.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An administering device comprising:
a surface element having an exterior surface forming at least a portion of a surface of the administering device, said surface element comprising an opening; and
an activation element for activating a priming of the device, wherein the activation element is generally in the opening and one of flush with or inset relative to the exterior surface.

2. The administering device according to claim 1, wherein the activation element is flush with or inset relative to the exterior surface when the activation element has not yet been activated.

3. The administering device according to claim 2, wherein the device has two ends and a longitudinal axis, the surface element comprising an inner wall extending generally from end to end and parallel to the longitudinal axis, the activation element shiftable in the longitudinal direction generally inside the inner wall.

4. The administering device according to claim 2, wherein the activation element is shiftable along an inner surface of the surface element.

5. The administering device according to claim 3, wherein the activation element comprises a sleeve having a longitudinal axis generally coaxial with the longitudinal axis of the administering device or parallel to it, said sleeve coupled to a priming rod such that the priming rod can be moved by the activation element to effect a priming.

6. The administering device according to claim 5, wherein the surface element is a setting ring for setting an amount of a substance to be administered.

7. The administering device according to claim 6, wherein the activation element is at an end of the administering device.

8. A device for administering, delivering or dispensing a substance, the device comprising:
a surface element forming at least portion of a surface of the device and defining an opening; and
a priming mechanism for priming the device, wherein the priming mechanism comprises an activation element for activating a priming procedure, wherein the activation element is generally in the opening of the surface element and is one of flush with or inset relative to the surface of the surface element.

9. The device according to claim 8, wherein the activation element comprises a surface one of flush with or inset relative to the surface of the surface element.

10. The device according to claims 9, wherein the activation element further comprises an inside coupling hub operably coupled to a priming rod such that the priming rod can be moved by the activation element to effect a priming.
